(19) United States
(12) Patent Application Publication
(10) Pub. No.: US 2022/0112461 A1
(43) Pub. Date: Apr. 14, 2022

(54) SHAKE FLASK GROWTH OF IMMUNE CELLS

(71) Applicant: CENTRE FOR COMMERCIALIZATION OF REGENERATIVE MEDICINE, Toronto (CA)

(72) Inventors: Danylo SIRSKYJ, Toronto (CA); Calley HIRSCH, Toronto (CA)

(21) Appl. No.: 17/416,352

(22) PCT Filed: Dec. 19, 2019
(86) PCT No.: PCT/CA2019/051864

§ 371 Date: Jun. 18, 2021

Related U.S. Application Data
(60) Provisional application No. 62/782,719, filed on Dec. 20, 2018.

(51) Int. Cl. C12N 5/0783; C12M 1/00; C12M 3/06; C12M 1/24; C12M 1/26; C12N 15/86; C07K 14/725

(52) U.S. Cl. CPC C12N 5/0638; C12M 27/20; C12M 27/16; C12M 23/08; C12M 23/38; C12M 29/04; C12N 2740/15043; C12N 5/0646; C12N 15/86; C07K 14/7051; C12N 2501/515; C12N 2501/51; C12N 2501/2302; C12M 33/04

(57) ABSTRACT

Provided herein are systems and methods for culturing, activating, transducing and expanding immune cells in shaker flasks with agitation.

### CROSS-REFERENCE TO RELATED APPLICATIONS

This application claims priority to U.S. Provisional Application No. 62/782,719, filed Dec. 20, 2018, which is incorporated herein in its entirety.

### BACKGROUND

Cellular immunotherapies are a growing area of research and commercial development. Particular therapies, such as chimeric antigen receptor (CAR) and T cell receptor (TCR) cell therapies, revolve around ex vivo development of genetically engineered T cells designed to selectively target and kill specific cancer cell types and have found promising use as cancer treatments. For example, CAR T cells recognizing the tumor antigen CD19 have been approved by the FDA for treatment of leukemia and lymphoma. CARs against many other targets are being studied in clinical trials. However, generating enough genetically engineered cells for administration to patients remains an ongoing challenge in the field. Immune cells are particularly sensitive to early in vitro culture stresses following patient extraction limiting the options available for efficient engineering and commercial scale development of these therapies. Therefore, there is a growing need for improved immune cell culture methods compatible will activation, transduction and explanation for cellular immunotherapies.

### SUMMARY OF THE INVENTION

In a first aspect, provided herein is a method of culturing immune cells, the method comprising expanding a population of immune cells in a culture medium in a shake flask with orbital agitation. In some embodiments, the immune cells are primary immune cells or immune cells derived from pluripotent stem cells. In some embodiments, the immune cells are CD3+ T cells. In some embodiments, the immune cells are CD4+ T cells, CD8+ T cells, or a combination thereof. In some the immune cells are Natural Killer (NK) cells. In some embodiments, the immune cells are obtained by leukapheresis or are obtained from whole blood. In some embodiments, the population of immune cells are expanded for at least 1 day.

In some embodiments, the cells are agitated at a speed between about 50 rpm and about 150 rpm. In some embodiments, the cells are agitated at a speed between about 60 rpm and about 100 rpm. In some embodiments, the cells are agitated at a speed between about 110 rpm and about 130 rpm.

In some embodiments, the method additionally comprises the step of activating the immune cells by culturing the immune cells in a culture medium. In some embodiments, the method additionally comprises transducing the immune cells with an expression vector. In some embodiments, the vector is a lentiviral vector. In some embodiments, the expression vector encodes for a chimeric antigen receptor (CAR) or a T cell receptor (TCR).

In some embodiments, the shake flask is a closed shake flask comprising a lid with an air inlet filter and a dip tube integrated into the lid to allow to removal of medium or cells without opening the shake flask. In some embodiments, the shake flask is open.

In some embodiments, the immune cells are T cells activated by culturing the T cells in a culture medium comprising anti-CD3 and anti-CD28 monoclonal antibodies.

In some embodiments, the population of immune cells is cultured for at least about 2 days. In some embodiments, the population of immune cells is cultured for at least about 3 days. In some embodiments, the culture medium is supplemented with IL-2 and human serum or serum replacement.

In a second aspect, provided herein is an immune cell culture system comprising a population of immune cells and a culture medium in a shake flask, the shake flask being secured to a platform of an orbital shaker which agitates the shake flask at a speed sufficient to prevent the immune cells from settling to the bottom of the shake flask. In some embodiments, the immune cells are primary immune cells. In some embodiments, the immune cells are CD3+ T cells. In some embodiments, the immune cells are CD3+, CD4+ T cells. In some embodiments, the immune cells are CD3+, CD8+ T cells. In some embodiments, the immune cells are Natural Killer (NK) cells. In some embodiments, the medium is supplemented with IL-2 and human serum or serum replacement.

In some embodiments, the shake flask is a closed shake flask comprising a lid with an air inlet filter and a dip tube integrated into the lid to allow to removal of medium or cells without opening the shake flask. In some embodiments, the shake flask is open. In some embodiments, diameter of the mouth of the shake flask is narrower than the diameter of the bottom of the shake flask. In some embodiments, the shake flask has at least one baffle.

### BRIEF DESCRIPTION OF DRAWINGS

The patent or patent application file contains at least one drawing in color. Copies of this patent or patent application publication with color drawings will be provided by the Office upon request and payment of the necessary fee.

The invention will be better understood and features, aspects, and advantages other than those set forth above will become apparent when consideration is given to the following detailed description thereof. Such detailed description will make reference to the following drawings.

FIG. 1 shows a photo of a shake flask with closed system attachments.

FIGS. 2A-2B shows growth and characterization of T cells in shake flasks compared to static culture. (FIG. 2A) Activated T cells grown in shake flasks at 90 rpm demonstrate similar fold expansion rates and viability after 5 days of culture (n=3 donors). (FIG. 2B) Phenotypes of T cells grown for 5 days in shake flasks at 90 rpm relative to static T-flask controls across three experiments.

FIGS. 3A-3B show growth and characterization of T cells in shake flasks at 90 rpm, 60 rpm, 30 rpm, in gas permeably bags or in static culture T-flasks. (FIG. 3A) Activated T cells grown in shake flasks at 60 rpm out performed those grown at 90 rpm and standard T flask static controls (n=1 donor). (FIG. 3B) T cell phenotypes from cultures grown for 5 days at 60 rpm or 90 rpm compared to T-flask controls (n=1 donor).

### DETAILED DESCRIPTION OF THE INVENTION

All publications, including but not limited to patents and patent applications, cited in this specification are herein incorporated by reference as though set forth in their entirety in the present application.

The present disclosure describes methods and systems for culturing immune cells with agitation. A population of immune cells is cultured in a shake flask with agitation for a time and under conditions sufficient to activate, transduce or expand the population of immune cells. Immune cells grown in shake flasks with agitation using the methods described herein are useful for any number of therapeutic applications known in the art. For example, chimeric antigen receptor (CAR) T cells grown using the methods described herein are useful for cancer immunotherapy treatments, such as CD19 CAR T cells for the treatment of leukemia and lymphoma. It is understood in the art that immune cells are sensitive to early culture shear stresses and are traditionally grown in static culture requiring open user manipulation for culture sampling, dilution, activation, and cell engineering increasing the risk of contamination of the culture. Using the methods described herein, immune cells grown in shake flasks with agitation can be sampled, diluted, activated, transduced, and modified in a closed system without exposure of the cells to the external environment.

In some embodiments, provided herein are methods for culturing and expanding a population of immune cells in a shake flask with agitation for a time and under conditions sufficient for the growth and expansion of the cells. The population of immune cells is cultured in a shake flask with agitation for about 1 day, about 2 days, about 3 days, about 4 days, about 5 days, about 6 days, about 7 days, about 8 days, about 9 days, about 10 days, about 11 days, or about 12 days. In some embodiments, the population of immune cells is cultured in a shake flask for between about 1 day and about 12 days, between about 2 days and about 10 days, between about 3 days and about 8 days, or between about 4 days and about 6 days. In some embodiments, the population of immune cells is transduced with an expression vector prior to or during culturing in the shake flask with agitation. In some embodiments, the population of immune cells is transduced with an expression vector prior to or during culture in the shake flask without agitation. In some embodiments, prior to transduction, the population of immune cells is activating using an appropriate activation method. The immune cells may be activated in the shake flask or may be activated in static culture prior to introduction into the shake flask. In some embodiments, the full process from cell seeding and activation to the completion of expansion occurs over about 2 days to about 12 days.

A population of immune cells suspended in a culture medium is introduced into a shake flask at a density between about 0.25×10⁶ cells/ml and about 10×10⁶ cells/ml. In some embodiments, the population of cells are introduced into the shake flask at a concentration of about 1.5×10⁶ cells/ml (e.g., 0.5×10⁶ cells/ml, 1×10⁶ cells/ml, 1.5×10⁶ cells/ml, 2×10⁶ cells/ml, or 2.5×10⁶ cells/ml).

The culture medium used with the population of immune cells in the shake flask may be any culture medium known in the art suitable for survival and growth of the immune cells. Suitable culture medium are known and used in the art. For example, suitable culture medium includes, but is not limited to Xuri™ EM medium (GE Healthcare), CTS OpTmizer T Cell Expansion SFM (Thermo Fisher Scientific), ImmunoCult-XF T Cell Expansion Medium (STEMCELL Technologies), or X-VIVO 15 Serum-free Hematopoietic Cell Medium (Lonza). In some embodiments, the culture medium is supplemented with about 5% human serum (e.g., 1%, 2%, 3%, 4%, 5%, 6%, 7%, 8%, 9%, or 10% human serum). In some embodiments, the culture medium is supplemented with a serum replacement alternative such as about 2% CTS Immune Cell SR (e.g., 1%, 2%, 3%, 4%, 5%, 6%, 7%, 8%, 9%, or 10% CTS Immune Cell SR). In some embodiments, the culture medium is supplemented with between about 50 IU/ml and about 500 IU/ml IL-2.

Immune cells suitable for use in the methods described herein include peripheral blood mononuclear cells, T cells, cytotoxic T lymphocytes, helper T cells, progenitor T cells, T regulatory cells (Tregs), Natural Killer (NK) cells, NK T cells, tumor infiltrating lympohcytes (TILs), dendritic cells (DCs), B-cells, and neutropils. The immune cells may be primary immune cells, immune cells derived from cord blood, immune cells derived from peripheral blood mononucleocytes, immune cells derived from bone marrow, pluripotent stem cell derived immune cells, induced pluripotent stem cell derived immune cells, a product from an allogeneic immune cell bank, or an immortalized immune cell line. In some embodiments, the immune cells are isolated from whole blood of a subject. In some embodiments, the immune cells are isolated from apheresis unites collected from a subject. In some embodiments, the immune cells are isolated from leukapheresis units collected from a subject. In some embodiments, the immune cells are CD3+ T cells. In some embodiments, the immune cells are CD3+, CD4+ T cells. In some embodiments, the immune cells are CD3+, CD8+ T cells. In some embodiments, the immune cells are a population of CD4+ T cells and CD8+ T cells. In some embodiments, the immune cells are CD14− or CD19− cells. In some embodiments, the immune cells are CD16+, CD56+NK cells.

In some embodiments, the immune cells are T cells. The T cells may be activated in the shake flask or may be activated in static culture and incubated prior to being seeding and introduced into the shake flask. T cells may be activated by any suitable means or method known in the art. Suitable means for activating T cells include, but are not limited to, exposure of the T cells to an anti-CD3 antibody, exposure of the T cells to an anti-CD28 antibody, exposure of the T cells to beads coated with anti-CD3/anti-CD28 monoclonal antibodies, exposure of the T cells to autologous antigen presenting cells (APCs), and exposure of the T cells to artificial APCs.

In some embodiments, the immune cells are NK cells. The NK cells may be activated in the shake flask or may be active in static culture and incubated prior to being seeded and introduced into the shake flask. NK cells may be activated by any suitable means or method known in the art. Suitable means for activating NK cells include, but are not limited to, exposure of the NK cells to an anti-CD335 antibody, exposure of the NK cells to an anti-CD2 antibody, exposure of the NK cells to beads coated with anti-CD335/anti-CD2 monoclonal antibodies, exposure of the NK cells to autologous antigen presenting cells (APCs), and exposure of the NK cells to artificial APCs.

In some embodiments, the immune cells may be genetically modified. Genetic modifications of the immune cells may include transduction with viral vectors or transfection with an expression vector for a specific receptor, marker, protein, mRNA, siRNA, or other heterologous expression product of interest. The genetic modification may be introduced by any suitable means known in the art. In some embodiments, the immune cells are transduced with a lentivirus that allows expression of a chimeric antigen receptor (CAR) or engineered T cell receptor (TCR).

Suitable TCRs are known in the art and will depend on the disease or disorder to be treated, including the specific type of cancer to be treated. T cells are engineered to express TCRs that recognize tumor specific antigens to selectively drive tumor cell elimination. For example, administration of modified T cells bearing a TCR that targets the NY-ESO-1 antigen have demonstrated promising clinical results across a range of malignancies, including non-small cell lung cancer, multiple myeloma, ovarian cancer and melanoma. A skilled artisan will understand that the methods described herein can be modified to engineer a wide variety of suitable TCR containing immune cells.

Suitable CARs will depend on the disease or disorder being treated, for example, the specific type of cancer to be treated. In one embodiment, the CAR may be specific to a tumor-specific antigen. CARs may be specific to a target antigen under clinical evaluation for CAR therapy as reviewed by Robyn A. A. Oldham & Jeffrey A. Medin (2017), Expert Opinion on Biological Therapy, DOI:10.1080/14712598.2017.1339687 (see, for example, Tables 3 and 4). Exemplary targets include, without limitation, CD19, BCMA, CD133, CD171, CD20, CD30, CD33, CEA, CLL1 (C—C Motif Chemokine Ligand 1), CLEC1A (C-Type Lectin Domain Family 1, Member A), EGFR, ERBB2, GD2, Kappa Ig (Igκ), IL13RA2, Mesothelin, MUC1, PSMA, and CD22.

In some embodiments, the immune cells are transduced with a vector comprising the genetic modification of interest. In some embodiments, the vector is a lentiviral vector. In some embodiment, the vector is a lentiviral vector which genetically engineers immune cells to expresses the CAR of interest in the immune cells. Other vectors and suitable CARs are known in the art and may be derived and are contemplated for use in the methods of the present disclosure.

Suitable vectors for use in the methods described are known in the art and contain the necessary elements in order for the gene encoded within the vector to be expressed in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the targeted protein. Another type of vector is a viral vector, wherein additional DNA segments may be ligated into the viral genome. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. Such vectors are referred to herein as “recombinant expression vectors” (or simply, “expression vectors” or “vectors”). In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses), which serve equivalent functions.

The vectors are heterologous exogenous constructs containing sequences from two or more different sources. Suitable vectors include, but are not limited to, plasmids, expression vectors, lentiviruses (lentiviral vectors), adeno-associated viral vectors (AAV) among others and includes constructs that are able to express a CAR (e.g., CD19 CAR) or a genetic modification of interest. A preferred vector is a lentiviral vector. Suitable methods of making lentiviral vector particles are known in the art. While specific lentiviral vectors have been used in the examples, the vectors are not limited to these embodiments and any lentiviral vectors or other vectors capable of expressing the CAR are contemplated for use in the practice of the current invention.

A vector can preferably transduce, transform or infect a cell, thereby causing the cell to express the nucleic acids and/or proteins encoded by the vector (e.g., the CAR or genetic modification of interest).

In a one embodiment, T cells are isolated from a leukapheresis product and activated with ImmunoCult T cell CD2/CD28/CD3 Activator. The activated T cells are then placed in a shake flask at a concentration between 1×10⁶-2×10⁶ cells/ml in culture medium supplemented with 5% human serum and IL-2 and cultured for about 1 day at 37° C. with 5% CO₂. After culturing for about 1 day with agitation in a shaker flask, activated T cells are then transduced by direct introduction of a lentivirus encoding a CAR and cultured for about 1 day. After culturing the activated and transduced T cells for about 1 day, the population of T cells is expanded in culture in the shaker flaks with agitation for about 2 days or about 3 days until a viable cell density (VCD) suitable for seeding in a 1 L bioreactor is achieved.

As used herein, “shake flask” refers to a hard walled vessel designed to hold a population of cells and at least 5 ml of a culture medium in which the culture medium does not spill or splash out of the shake flask when agitated at a speed sufficient to prevent the cells from settling to the bottom of the shake flask. The shake flask may be a commercially available shake flask, such as a conical or an Erlenmeyer-type flask. The shake flask may be made of any suitable material, such as but not limited to glass, plastic, polycarbonate, polypropylene, polystyrene, or another biocompatible polymer. The shake flask may be of any volume suitable to contain the medium and population of cells without spilling during agitation. The volume of the shake flask may be, but is not limited to 125 ml, 250 ml, 500 ml, 1 L, or 2 L. In some embodiments, the shake flask has a round bottom, conical walls, a neck and an open top, the diameter of the open top being narrower than the diameter of the bottom. In some embodiments, the shake flask may include one or more baffles. In some embodiments, the top of the shake flask is closed. In a preferred embodiment, the shake flask is a 125 ml, 250 ml, 500 ml, 1 L, or 2 L conical flask.

In some embodiments, the shake flasks are agitated using an orbital shaker at a speed sufficient to prevent cell settling in the shake flask and minimize cell clumping. In general, the cell viability should remain at least 80%. In some embodiments the shake flasks are agitated at 30 rpm, 40 rpm, 50 rpm, 60 rpm, 70 rpm, 80 rpm, 90 rpm, 100 rpm, 110 rpm, 120 rpm, 130 rpm, 140 rpm, or about 150 rpm. In some embodiments, the shake flask is agitated at a speed between about 50 rpm and about 150 rpm, between about 60 rpm and about 130 rpm, about 70 rpm and about 120 rpm, between about 80 rpm and about 110 rpm, or between about 90 rpm and about 100 rpm. In some embodiments, the shake flask is agitated at between about 60 rpm and about 90 rpm. In some embodiments, the shake flask is agitated at between about 90 rpm and about 130 rpm. A skilled artisan will appreciate that smaller shake flasks, as measured by volume, will require slower speeds. In some embodiments, the shake flask is a 125 ml shake flask agitated at about 60 rpm to about 90 rpm. In some embodiments, the shake flask is a 1 L shaker flask agitated at about 90 rpm to about 120 rpm. The shake flask may be agitated continuously or may be agitated intermittently.

In some embodiments the shake flask is attached or secured to the platform of the orbital shaker. In some embodiments, the bottom of the shake flask is parallel to the platform of the orbital shaker. The shake flask and the orbital shaker may be in any configuration suitable for the growth and survival of the immune cells during agitation.

In some embodiments, the shake flasks are closed shake flasks sealed with a lid or closure system that does not allow for the passage of liquids or solids without manipulation by the user. Suitable seals and lids are known in the art. The closed shake flask may be a closed-system shake flask including a large air inlet filter and built in dip tube to allow removal or exchange of medium or cells without opening the shake flask. In some embodiments, the shake flasks are left open or manually closed such as with a vented cap that needs to be removed prior to removal or exchange of cells and medium.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates successful activation, transduction and expansion of T cells in shake flask cultures.

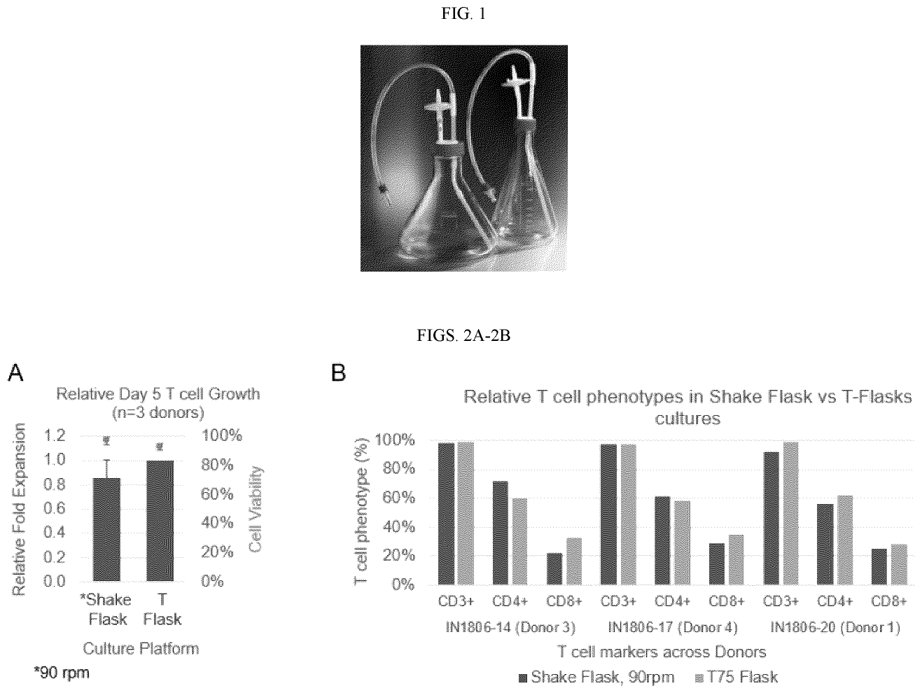

Primary human T cells were isolated from Leukopacks (HemaCare) and activated with soluble CD3/CD28/CD2 ImmunoCult T Cell Activator. Following activation, the T cells were placed into an Erlenmeyer-type shake flask, such as those depicted in FIG. 1, at a density of 1×10⁶-2×10⁶ cells/ml. Activated T cells were then cultured with agitation in the shake flasks for about 1 day in Xuri EM (GE Healthcare) culture medium supplemented with 5% human serum and IL-2. After 1 day of culturing with agitation, the T cells are transduced by addition of viral vectors encoding a CAR. The transduced, activated T cells were then cultured with agitation in the same medium for about 1 day. For days 2 through 4 or 5, the activated, transduced T cells are cultured with agitation in the shake flasks at 37° C. with 5% CO₂ until VCD sufficient for seeding in a 1 L bioreactor is achieved.

As shown in FIG. 2A, activated T cells grown in shake flasks with agitation at 90 rpm demonstrate similar fold expansion rates and viability after 5 days of culture as cells grown in static culture. The phenotypes of the cells grown with agitation were similar to the phenotypes of T cells grown in static culture (FIG. 2B).

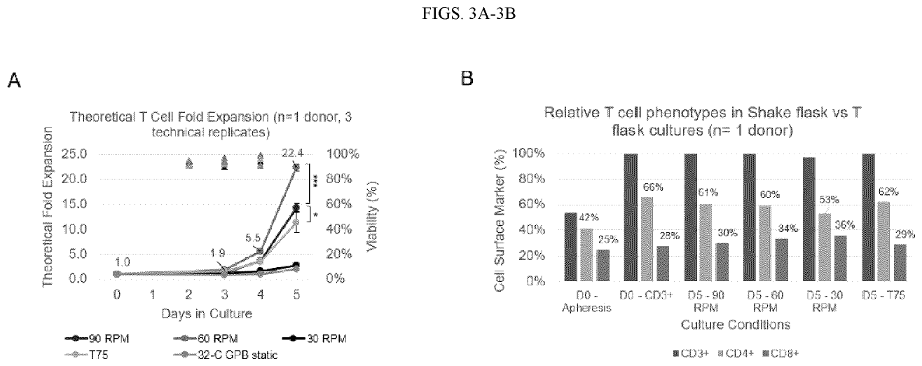

As shown in FIG. 3A, activated T cells grown in shake flasks with agitation at 60 rpm outperformed T cells grown in shake flasks with agitation at 90 rpm and outperformed growth of T cells in static culture. The T cell phenotypes of cells grown in shake flasks with agitation at either 90 or 60 rpm is similar to the phenotypes of T cells grown in static culture (FIG. 3B).

We claim:

1. A method of culturing immune cells, the method comprising:
expanding a population of immune cells in a culture medium in a shake flask with orbital agitation.

2. The method of claim 1, wherein the immune cells are primary immune cells or immune cells derived from pluripotent stem cells.

3. The method of claim 1, wherein the immune cells are CD3+ T cells.

4. The method of claim 1, wherein the immune cells are CD4+ T cells, CD8+ T cells, or a combination thereof.

5. The method of claim 1, wherein the immune cells are Natural Killer (NK) cells.

6. The method of claim 1, wherein the immune cells are obtained by leukapheresis or are obtained from whole blood.

7. The method of claim 1, wherein the population of immune cells are expanded for at least 1 day.

8. The method of claim 1, wherein the cells are agitated at a speed between about 50 rpm and about 150 rpm.

9. The method of claim 8, wherein the cells are agitated at a speed between about 60 rpm and about 100 rpm.

10. The method of claim 8, wherein the cells are agitated at a speed between about 110 rpm and about 130 rpm.

11. The method of claim 1, wherein the method additionally comprises the step of
activating the immune cells by culturing the immune cells in a culture medium.

12. The method of claim 11, additionally comprising transducing the immune cells with an expression vector.

13. The method of claim 12, wherein the vector is a lentiviral vector.

14. The method of claim 12, wherein the expression vector encodes for a chimeric antigen receptor (CAR) or a T cell receptor (TCR).

15. The method of claim 1, wherein the shake flask is a closed shake flask comprising a lid with an air inlet filter and a dip tube integrated into the lid to allow to removal of medium or cells without opening the shake flask.

16. The method of claim 1, wherein the shake flask is open.

17. The method of claim 11, wherein the immune cells are T cells activated by culturing the T cells in a culture medium comprising anti-CD3 and anti-CD28 monoclonal antibodies.

18. The method of claim 1, wherein the population of immune cells is cultured for at least about 2 days.

19. The method of claim 18, wherein the population of immune cells is cultured for at least about 3 days.

20. The method of claim 1, wherein the culture medium is supplemented with IL-2 and human serum or serum replacement.

21. An immune cell culture system comprising a population of immune cells and a culture medium in a shake flask, the shake flask being secured to a platform of an orbital shaker which agitates the shake flask at a speed sufficient to prevent the immune cells from settling to the bottom of the shake flask.

22. The system of claim 21, wherein the immune cells are primary immune cells.

23. The system of claim 21, wherein the immune cells are CD3+ T cells.

24. The system of claim 21, wherein the immune cells are CD3+, CD4+ T cells.

25. The system of claim 21, wherein the immune cells are CD3+, CD8+ T cells.

26. The system of claim 21, wherein the immune cells are Natural Killer (NK) cells.

27. The system of claim 21, wherein the medium is supplemented with IL-2 and human serum or serum replacement.

28. The system of claim 21, wherein the shake flask is a closed shake flask comprising a lid with an air inlet filter and a dip tube integrated into the lid to allow to removal of medium or cells without opening the shake flask.

29. The system of claim 21, wherein the shake flask is open.

30. The system of claim 21, wherein diameter of the mouth of the shake flask is narrower than the diameter of the bottom of the shake flask.

31. The system of claim 21, wherein the shake flask has at least one baffle.
